(19) United States
(12) Patent Application Publication
(10) Pub. No.: US 2026/0120893 A1
(43) Pub. Date: Apr. 30, 2026

(54) Use of Artificial Intelligence and Machine learning capability in Medical Diagnosis and Treatment to reduce stress on medical personal and improve treatment efficiency

(71) Applicants: Mammen Thomas, Seattle, WA (US); Arun Mammen Thomas, Dublin, CA (US)

(72) Inventors: Mammen Thomas, Seattle, WA (US); Arun Mammen Thomas, Dublin, CA (US)

(21) Appl. No.: 19/312,896

(22) Filed: Aug. 28, 2025

Related U.S. Application Data
(63) Continuation-in-part of application No. 19/179,944, filed on Apr. 15, 2025.
Application No. 19/179,944 is a division of application No. 18/930,973, filed on Oct. 29, 2024, now Pat. No. 12,437,881.

(51) Int. Cl. G16H 80/00; G06F 40/58; G06T 17/00; G16H 10/20; G16H 50/20

(52) U.S. Cl. CPC G16H 80/00; G06F 40/58; G06T 17/00; G16H 10/20; G16H 50/20

(57) ABSTRACT

A system and process for screening of patients, collection of medical data, and arriving at a preliminary medical diagnosis and treatment plan for a patient using Artificial Intelligence (AI) with machine learning (ML) capability is disclosed. Such a system and method will be usable to reduce the load on the medical personal in hospitals and medical centers/medical offices, enabling efficient use of available time of medical professionals who are a scarce resource in many parts of the world.

### FIELD OF INVENTION

The invention relates to automated generation of a preliminary diagnosis and identification of a preliminary treatment plan for patients without involvement of medical professionals/doctors using the capabilities of Artificial intelligence and Machine learning with natural language recognition ability and diagnostic ability to save time the doctors have to spend diagnosing the problems of patients ang generating a treatment plans.

### DESCRIPTION OF FIGURES

FIG. 1, (100) is a depiction of an embodiment of a system of the present invention enabling a patient at a clinic interaction in natural language with the Artificial Intelligence (AI) with machine learning (ML) capability to enable collection of data relating to the patient and his illness.

FIG. 2, (200) is an exemplary natural language understanding and question generation system with AI and ML capability for understanding from the patient, the patient's problem and using inputs from a continuously updating medical database define tests to be done to use with the patients inputs to arrive at a preliminary diagnosis and a preliminary treatment plan for the patient to be included in a report provided to the medical personal.

FIG. 3, (300) is an exemplary flow chart of the use of the system for collecting personal data and medical history of a patient, the system having AI with ML capability enabled to generate a preliminary diagnosis and a preliminary treatment plan for the patient without involving medical professionals/doctors, which is provided to the doctors to review and finalize.

### BACKGROUND

In many regions of the world, especially in rural settings the availability of medical personal such as nurses and doctors are limited. In a medical facility covering a large geographical area there may exist only one medical center with a limited number of medical personal. These medical personal are expected to provide medical help to a substantial number of patients, which put a stain on them. Even for developed nations like USA the lack of medical help in rural setting is becoming a problem. A solution is currently needed to improve the efficiency of the medical staff in these locations to cater to the population covered by them.

Currently there exist capabilities using the web, skype and other internet related connectivity for medical personal to interact with a patient from a remote site. But the medical personal is fully engage in the data collection during this interaction and their load is not reduced and their efficiency to cater to the population is not improved by this capability.

The prior art of record applications Ser. Nos. 18/930,973 and 19/179,944 cover a system where a patient in a remote location is able to interact with the AI with ML capability that is able to create an understanding of the patients problem and provide a preliminary diagnosis and treatment plan to be provided to medical personal in a primary clinic for review and finalization. The medical personal are then able to discuss with the patient and refine the diagnosis and treatment plan over the web or a virtual reality environment to finalize the diagnosis and treatment plan based on the preliminary diagnosis and treatment plan. If the review finds additional test needs and hospitalization, the patient has to be transported to a test facility and hospital This is mainly a need in remote locations where a patient has no immediate access to a medical facility.

### DESCRIPTION

A system and process for screening of patients, collection of medical data, and arriving at a preliminary medical diagnosis and treatment plan for a patient using Artificial Intelligence (AI) with machine learning (ML) capability is disclosed. Such a system and method will be usable to reduce the load on the medical personal in hospitals and medical centers/medical offices, enabling efficient use of available time of medical professionals who are a scarce resource in many parts of the world.

The availability of artificial intelligence (AI) with machine learning (ML) capability provides a solution for improving the efficiency of patient care by reducing the load of the medical staff by offloading the operations of personal and medical data collection in natural language from the patient. The AI with ML capability is then enabled to create an understanding of the patient's illness and provide test requirements to be completed and collected to generate from the collected data and test results a preliminary diagnosis and treatment plan for the patient's illness. The generated data and test results as well as the collected medical data is used by a diagnostic unit connected to the AI with ML capability, with inputs from a medical data base that is continuously updating from external inputs, to generate a preliminary diagnosis and a preliminary treatment plan for the patient. This is provided to the medical staff, or doctors to review, ask further questions and provide additional tests which will enable them to arrive at a final diagnosis of the patient's illness and create a final treatment plan.

### DETAILED DESCRIPTION

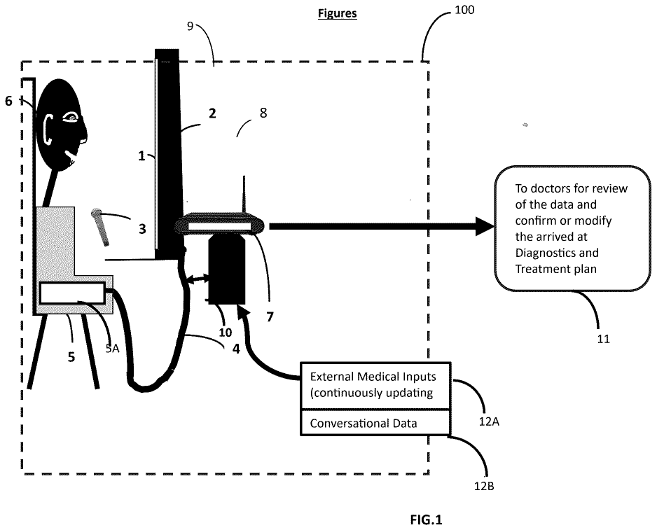

FIG. 1, 100, shows a patient interview room 9 with a patient 6 having an illness sitting on a chair 5 in front of a natural language input/output (I/O) system comprising a video screen 2 and speaker 1, and having an associated microphone 3. The I/O system connected to a first console 5A, that is a system console, using a cable 4. The s first console 5A comprising at least a processor providing processing capability to the system, a natural language input understanding capability, a query or question generation capability, an AI with ML capability, an automatic diagnostic unit, and a natural language output unit. A second console, that is a memory console 10, holds all the memories needed for the storage of data and includes a plurality of data bases comprising an updating 12B conversational data DB, a Stored Language DB, a Learned Questions or Queries and Answers DB, a learned Diagnosis and Treatment plan DB, a continuously updating 12A clinical/Medical knowledge DB. The system shown in FIG. 1 also has a communication module 7 that can transfer data to Doctors over a wireless 8 connection. Even though the wireless connection is preferred it is not meant to be limiting. Other connections such as wired or blue tooth may be employed to transfer data to doctors for review, modify or approve the diagnosis and the treatment plan as shown 11.

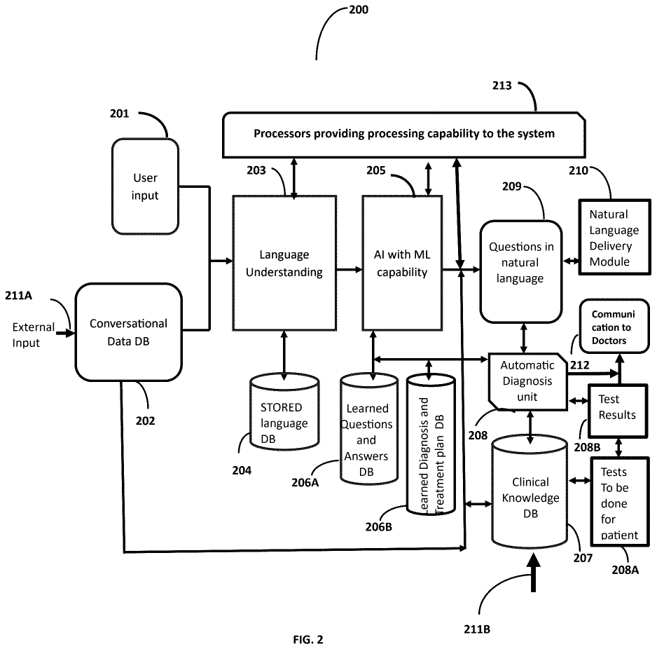

FIG. 2, 200, is an exemplary natural language understanding and question generation system with AI and ML capability for understanding from the patient, the patient's problem and using inputs from a continuously updating medical database define tests to be done to use with the patients inputs to arrive at a preliminary diagnosis and a preliminary treatment plan for the patient to be provided to the medical personal. It is used to describe the inventive idea in more detail using the modules in the system.

As shown in FIG. 2 the inputs from the patient are received by the user input module 201 which is coupled to a conversational data base (DB) 202 for natural language and is updated on a continuous basis via an external input 211A as natural language uses evolve. The user inputs 201 and the conversational data are used by a language understanding module 203 to convert the natural language inputs received into a medically understandable language format using the processing power of the processing capability 213 coupled to it. The converted medically understandable language is stored in a Stored language DB 204 for use by an artificial intelligence (AI) with machine learning(ML) capability 205 coupled to the language understanding module 203. The AI with ML capability 205 is trained to generate queries or questions, convert them to natural language in a questions in natural language module 209 and present it via a natural language delivery module 209 to the patient to answer. The patient's answers are input into the user input module 201 and processed as earlier. Additional questions are generated by the AI with ML capability 205 to collect all the patient's personal and medical data. The AI with ML capability 205 is coupled to a continuously updating 211B clinical knowledge DB 207 that allows it to generate additional questions to the patient to arrive at understanding of the medical problem or patient's illness as presented by the patient. The questions presented to the patient and the answers received are saved in a learned questions and answers DB 206A for enhancing the machine learning of the AI with ML capability module 205.

A test generation module 208A coupled to the clinical knowledge DB 207 and an automatic diagnostic unit 208 uses the understood problem or illness of the patient to generates tests for the patient to fully clarify patient's illness. The test results are collected y a test result module 208B coupled to the automatic diagnosis unit 208 and fed into the automatic diagnostic unit 208. The automatic diagnostic unit 208 uses the test results, the understood medical problem of the patient and the inputs from the medical knowledge DB 207 to generate a preliminary diagnosis and a preliminary treatment plan for the patient. This is stored in a learned diagnosis and treatment plan DB 206B for enhancing he ML capability of the AI with ML capability module 205. The information in all data bases should be understood to be linked to the patient information collected for generation of a report on the patient by the AI with ML capability 205. The report comprises the collected and stored data on the patient including personal information, medical history, the preliminary diagnosis and the preliminary treatment plan. This report is now sent over a communication channel by a communication module 212 of the system to the medical personal/doctors for review, and creation of additional test requirements for confirmation of diagnosis and finalization of the diagnosis and treatment plan for the patient. Where necessary the doctors may interact directly with the patient to discuss and clarify his problems, collect additional inputs and explain the final diagnosis and final treatment plan.

The use of the system of collect and consolidate the patient's personal and medical information and create the necessary report with preliminary diagnosis and preliminary treatment plan relieves the medical staff of a substantial portion of the load and saves the time the medical personal have to devote to each individual patient. This increases and improves the utilization of the efficiency of use of the available resources, including doctor's time spent with each patient, at the office.

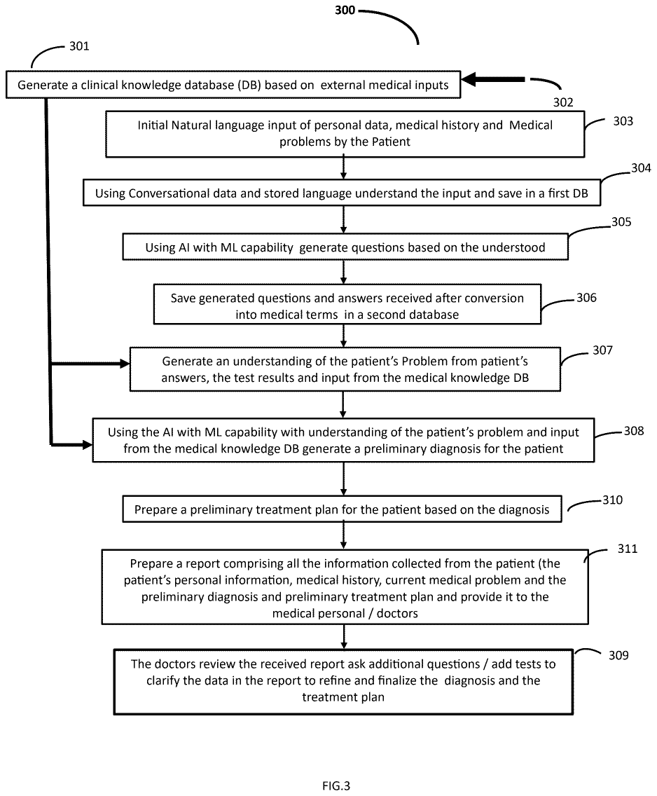

FIG. 3 300, is an exemplary and nonlimiting flow chart of the operation of the system to reduce the load on the medical staff at a clinic and improve the efficiency and utilization of available resources.
301—Generate a clinical Knowledge data base(DB) of all available data from medical sources.
302—Continuously update the clinical knowledge DB with inputs from all available external sources, such as medical libraries, medical peer reviewed papers, etc.
303—A patient with a medical problem, typically and illness arrives at the clinic. He is initially put in front of the system to input his details (Personal data and medical history) and his medical problem in a natural language.
304—Using conversational data from a conversational data DB and stored language from a stored language DB converts the user input into normally understandable and medically understandable language using the available processing power and save as updates in a first data base, the stored language DB, to enhance the learning of the Artificial Intelligence (AI) with machine learning (ML) capability.
305—The understandable inputs are provided to the AI with ML capability to generate additional questions (queries) to be presented to the patient for answering. These questions are based on prior understanding of the patient's inputs to enable collection of the patients personal and medical data and get a picture of his problem or illness.
306—The generated questions and answers are saved in a second database, the learned questions and answers DB, as updates to the data stored there and used to enhance the learning of the AI with ML capability.
307—From the set of inputs generate an understanding of the patient's medical problem. Using input from the clinical knowledge DB and the understanding of the patient's problem develop additional tests to be done by the patient to and generate a report of the individuals personal and medical data and problem as presented by him supported by the test results.
308—Using an automatic diagnostic unit coupled to the AI with ML capability, the test results and the understanding of the patient's medical problem develop a preliminary diagnosis for the patient.
309—Using the automatic diagnostic unit and the developed diagnosis, generate also a preliminary treatment plan for the patient. Save the generated preliminary diagnosis and the preliminary treatment plan in a learned diagnosis and treatment plan DB.
310—Update the patient report with the preliminary diagnosis and preliminary treatment plan and provide it to the medical personal/doctors at the clinic for review.
311—The doctors review the provided data and report and add additional tests to arrive at a final diagnosis and an associated treatment plan for the patient.

The system as described is able to off load the operation of collecting patient's personal and medical data and also defining tests to arrive at an initial diagnosis for the patient. By generating a preliminary report comprising these to be provided to the doctors the system is able to reduce the time spent by the doctors and other medical personal spend over 50% of time in data collection and reporting and less than 50% of time with patients. By off lading even a portion of the load and time spent (time pressure) on the doctors and other medical staff spent for collection of data and report preparation will enable the doctors and medical personal more time to cater to the patients and improve the efficiency of the clinical set up. This will also improve the quality of care by improving the utilization of scares resources and improve their efficiency of patient care.

### From Chat ZGPT Support for Load on Doctors

“A study revealed that physicians spend 49% of their time on Electronic Health Records and other desk work, and only 27% on direct clinical face time with patients. This means about half their work is related to data collection and management, and a quarter to direct patient interaction.”

Even though the inventive idea is described here using a specific embodiment, it is not meant to be limiting. There will be many changes that can be implemented to the embodiment that will achieve the same result as will be well known to practitioners of the art. These are covered by the current application.

1. to 13. (canceled)

14. A system for reducing a time pressure on medical professional and doctors and improve their efficiency of patient care, the system comprising:
a patient in a doctors'office in front of an input/output (I/O) module;
the I/O module comprising a user input module and a natural language delivery module, in the doctors'office;
the user input module comprising at least a microphone coupled to a video terminal;
the natural language delivery module comprising at least a speaker and the video terminal;
a system console comprising a processing capability sufficient for the operation of the system including an Artificial intelligence (AI) with a Machine learning (ML) capability;
a memory console comprising all the data bases required for the system operation;
the system receives natural language inputs from the patient via the user input module, generate additional questions and presents them to the patient via the natural language delivery module, to be answered by the patient, the questions answers to better understand the patient's current medical problem as presented by the patient and understand the patient's personal data and information, and also the patient's medical history;
the system using AI with ML capability and the information in the data bases generates additional tests for the patient to complete to further understand and confirm the patient's current medical problem;
the system based on the understood and confirmed current medical problem of the patient generates a preliminary diagnosis and a preliminary treatment plan for the patient's current medical problem;
the system using the AI with ML capability further generates a report comprising the patient's personal data and information, patient's medical history, the patient's current medical problem as understood, the preliminary diagnosis and the preliminary treatment pan for the patient based on the understood current medical problem of the patient and provides the report to the doctors;
wherein the report generated and provided to the doctors enable a reduction in a load and the time pressure on the doctors at the doctors'office, thereby improving their efficiency of patient care.

15. The system of claim 14, wherein the system console comprises sufficient processing capability for all operations of the system including for the AI with the ML capability.

16. The system of claim 14, wherein the I/O module of the system receives and using the system capabilities process the natural language inputs into medically understandable language.

17. The system of claim 16, wherein the system uses the processing capability available in the console to process the natural language inputs into medically understandable language in a language understanding module coupled to the user input module and using inputs from a conversational data base that is continuously updated via an external input.

18. The system of claim 14, wherein the system console comprises the AI with ML capability coupled to a clinical knowledge database and an automatic diagnosis unit;
wherein the clinical knowledge database is continuously updated from external sources of medical information; and
wherein the AI with ML capability is further coupled to a questions generation capability in natural language module;
wherein the AI with ML capability is used to generate questions to be presented to the patient in natural language for answers by a natural language delivery module;
the answers to the generated questions to further improve the understanding of the patient's current medical problem.

19. The system of claim 18, wherein the automatic diagnosis unit specifies additional tests to be done by the patient to confirm the understanding of the patient's current medical problem and develop a preliminary diagnosis and a preliminary treatment plan for the patient.

20. The system of claim 14, wherein the memory console comprises the conversational database and a stored language database both coupled to the language understanding module.

21. The system of claim 14, wherein the memory console holds all the data databases of the system;
wherein the databases of the system comprise: the conversational data base and a stored language data base, both coupled to the language understanding module, a learned questions and answers database coupled to the AI with ML capability, a learned diagnosis and treatment plan database coupled to the automatic diagnosis unit, the AI with ML capability and the clinical knowledge database.

22. A system for reducing the time pressure on medical professionals and doctors and improve their efficiency of patient care, the system comprising:
a patient interview room comprising a patient chair, in front of a natural language input/output (I/O) module;
a first system console and a second memory console;
wherein the system console comprises at least a processor providing all the processing power needed for the system operation, a natural language input with natural language understanding capability, a query or question generation capability coupled to an AI with ML capability, an automatic diagnostic unit also coupled to the AI with ML capability, and a natural language output connected to the I/O system;
wherein the memory console comprises a plurality of data bases (DB)s;
the DBs comprising a conversational data DB, a Stored Language DB, a Learned Questions or Queries and Answers DB, a learned Diagnosis and Treatment plan DB, a clinical/Medical knowledge DB;
the system receives natural language inputs from a patient via the I/O module, generates questions, using the question generation capability, to be answered by the patient to provide an understanding of the patient's current medical problem, generates tests for the patient to complete, the results of which provide for further understanding and confirmation of the patient's current medical problem, generates a report comprising the patient's personal data and information, patient's medical history and the understood and confirmed patient's current medical problem;
the report further including a preliminary diagnosis and a preliminary treatment pan for the patient based on the understood and confirmed medical problem of the patient to be provided to the doctors for review and finalization of the diagnosis and treatment plan;
wherein the report generated and provided to the doctors enable a reduction in a load and the time pressure on the doctors at a medical clinic, thereby improving their efficiency of patient care.

23. The system of claim 22, wherein the natural language inputs are converted to a medically understandable language inputs using a language understanding module coupled to the at least the processor providing all the processing power needed for the system, the conversational data DB, and a stored language DB.

24. The system of claim 23, wherein the AI with ML capability coupled to, the at least the processor, the AI with ML capability accepts the converted understandable inputs and using the inputs from the learned questions and answers DB, generate questions to be presented to the patient;
wherein the answers to the questions enable further understanding of the patient's medical problem.

25. The system of claim 24, wherein the automatic diagnostic unit uses the AI with ML capability, the understood patient's problem and input from the clinical knowledge DB, to generate tests in the test generation module;
wherein the tests are to be completed by the patient and the results provided to the test results module to be provided to the automatic diagnostic module; and
wherein the automatic diagnostic module is able to use the AI with ML capability, the understood patient's problem, and input from the clinical knowledge DB, and the received test results to arrive at the preliminary diagnosis for the patient's medical problem.

26. The system of claim 25, wherein the automatic diagnostic unit is configured, to generate the preliminary treatment plan based on the preliminary diagnosis of the patient's medical problem.

27. The system of claim 26, wherein the system using the AI with ML capability generates a report regarding the patient comprising the collected and available personal data, medical history of the patient, the preliminary diagnosis and the preliminary treatment plan.

28. The system of claim 26, wherein the generated report is sent over a communication channel by a communication module of the system to the medical personal/doctors for review and finalization of the diagnosis and treatment plan.

29. The system of claim 22, wherein the conversational data DB is updated from external input.

30. The system of claim 22, wherein the clinical/Medical knowledge DB is continuously updated from inputs from all available external sources, such as medical libraries, medical peer reviewed papers, etc.

31. A method of using a system comprising an artificial Intelligence (AI) with machine learning (ML) capability, a natural language input-output (I/O) capability and a processing capability for reducing the time pressure on medical professional and doctors and improving their efficiency of patient care at a medical clinic, the method comprising;
collecting inputs in natural language from a patient having a medical problem using an I/O module;
wherein the inputs comprise personal data, medical history and patient's inputs regarding the patient's medical problem;
converting the natural language input to a language that is medically understandable using the processing capability of an at least a processor coupled to a language understanding module;
generating gestions to be presented to the patient for answers based on the inputs received using a question generation module coupled to an AI with ML capability;
wherein answers to the presented questions from the patient enable an initial understanding of the patient's current medical problem;
generating tests by a test generation module, coupled to the AI with ML capability, based on the initial understanding of the patient's current medical problem from the patient's initial inputs, answers to questions and using input from a continuously updating clinical database;
wherein the tests are to be completed by the patient and test results provided to a test result module;
wherein the test results are used to further understand and confirm the initial understanding of the patient's current medical problem;
generating by an automatic diagnosis unit, using the confirmed initial understanding of the patient's current medical problem and inputs from the continuously updating clinical database a preliminary diagnosis and a preliminary treatment plan for the patient;
preparing a report comprising the patient's personal data, the patient's medical history, the preliminary diagnosis and the preliminary treatment plan for the patient;
providing the prepared report to the doctors and medical staff at the medical clinic, thereby reducing the time pressure on the medical professional and doctors and improving their efficiency of patient care.

32. The method of claim 31, wherein the report comprising the patient's personal data, the patient's medical history, the preliminary diagnosis and the preliminary treatment
